SEQUENCE LISTING (1 sequence)

SEQ ID NO: 1 - protein, 14 aa, Mycoplasma fermentans
  MALP-2 (1)..(14): 2,3-Diacylglyceryl-cysteinyl-peptide or 2,3-Diacylglyceral-serinyl-peptide
XGNNDESNISFKEK

(19) United States
(12) Patent Application Publication
(10) Pub. No.: US 2004/0127405 A1
(43) Pub. Date: Jul. 1, 2004

(54) Methods for treating lung infections and lung tumors and for treating and preventing lung metastases

(76) Inventors: Peter Muhlradt, Braunschweig (DE); Anke Luhrmann, Hannover (DE); Thomas Tschernig, Hildesheim (DE); Reinhard Pabst, Hannover (DE)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/412,547

(22) Filed: Apr. 11, 2003

Related U.S. Application Data
(63) Continuation-in-part of application No. 10/398,094.
Application No. 10/398,094 is a 371 of international application No. PCT/EP01/11414, filed on Oct. 2, 2001.

(30) Foreign Application Priority Data
Oct. 2, 2000 (DE) 10048804.4

(51) Int. Cl. A61K 38/17; A61K 38/10; C07K 14/775

(52) U.S. Cl. 514/12; 514/14; 530/359

(57) ABSTRACT

The invention relates to methods of treating lung infections and lung tumors and treating and preventing metastases of extrapulmonary tumors by administering lipopeptides or lipoproteins having the following formula (I):

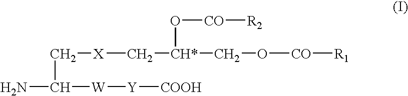

wherein:

R₁ and R₂, which may be the same or different from one another, denote C₇₋₂₅ alkyl, C₇₋₂₅ alkenyl, or C₇₋₂₅ alkynyl,

X denotes S, O, or CH₂,

W denotes CO or S(O)ₙ (where n=1 or 2),

Y denotes a physiologically acceptable amino acid sequence, and

* denotes an asymmetric carbon atom.

### CROSS REFERENCE TO RELATED APPLICATION

This is a continuation-in-part of U.S. Ser. No. 10/(serial number not available) filed Apr. 2, 2003, which is the U.S. national phase of International Application No. PCT/EP01/11414, filed Oct. 2, 2001, the entire disclosure of which is hereby incorporated herein by reference.

### BACKGROUND OF THE INVENTION

1. Technical Field

The invention relates to methods for treating lung infections and lung tumors, for treating and preventing lung metastases, for preventing lung inflammations, and for increasing lymphatic tissue in the bronchial mucosa, by administering lipopeptides or lipoproteins.

2. Brief Description of Related Technology

It is known that lipopeptides, which were originally isolated from micro-organisms and which are available synthetically, activate macrophages (Baschang, C. 1989. Tetrahedron 45:6331-6360.). Variants of such lipopeptides from mycoplasmas are, by virtue of particular structural features, especially active (Mühlradt, P. F., M. Kieβ, H. Meyer, R. Süβmuth, and G. Jung. 1997. J. Exp. Med. 185:1951-1958; Mühlradt, P. F., M. Kieβ, H. Meyer, R. Süβmuth, and G. Jung. 1998. Infect. Immun. 66: 4804-4810). Such lipopeptides are obtainable synthetically (See, for example, Metzger, J. W., K-H. Wiesmüller and G. Jung. 1991. Int. J. Peptide Protein. Res. 38: 545-554, the entire disclosure of which is hereby incorporated herein by reference). At picogram per milliliter (pg/ml) concentrations in cell cultures, they are capable of stimulating various cells, including primarily macrophages, to synthesize proinflammatory cytokines (interleukin-1, interleukin-6, tumor necrosis factor) and chemokines (MIP-1, M1P-2, MCP-1, IL-8) (Deiters, U. and P. F. Mühlradt. 1999. Infect. Immun. 67: 3390-3398; Kaufmann, A., P. F. Mühlradt, D. Gemsa and H. Sprenger. 1999. Infect. Immun. 67: 6303-6308).

It is known, furthermore, that the physiological receptors for such lipopeptides are the Toll-like receptors 2 and 6 and that the R stereoisomers of the lipopeptides are preferentially active (Morr, M., O. Takeuchi, S. Akira, M. M. Simon, and P. F. Mühlradt. 2002. Eur. J. Immunol. 32: 3337-3347).

It has hitherto not been suggested to use lipopetides therapeutically because the conventional bacterial lipoproteins are practically inactive in animal tests (Hauschildt, S., H. U. Beuscher, G. Jung, W. Bessier and A. Ulmer. 1994. FEMS Immunol. Med. Microbiol. 8:77-82). The macrophage-activating lipopeptide MALP-2 (an S-(2,3-bisacyloxypropyl)-cysteine peptide) isolated from Mycoplasma fermentans is, however, active in animal tests in the sense that intraperitoneal injection of a few μg has brought about leukocyte infiltration (Deiters, U. and P. F. Mühlradt. 1999. Infect. Immun. 67: 3390-3398).

It has hitherto not been possible, using non-toxic means, to achieve controlled infiltration of leukocytes into the lungs. Such controlled infiltration of leukocytes into the lungs can be desirable, for example, in order to combat chronic infections or for the therapy of inoperable tumors. A corresponding therapy in the treatment of bladder tumors consists in attracting in, and activating, macrophages by injecting living mycobacteria into the bladder (Zlotta A. R., J. P. Van Vooren, O. Denis, A. Drowart, M. Daffe, P. Lefevre, L. Schandene, M. De Cock, J. De Bruyn, P. Vandenbussche, F. Jurion, K. Palfliet, J. Simon, C. C. Schulman, J. Content, K. Huygen. 2000. Int. J. Cancer 87: 844-52). The inhalational administration of the lymphokine interleukin-2 results in the reduction of tumor metastases in the lungs with a low level of side-effects (Huland, E., H. Heinzer, H. Huland, R. Yung. 2000. Cancer J. Sci. Am. 6: Suppl.1, 104-112).

### BRIEF DESCRIPTION OF THE DRAWINGS

For a more complete understanding of the invention, reference should be made to the following detailed description and accompanying drawings wherein:

FIG. 1 shows the kinetics of the increase in the leukocyte count in bronchoalveolar lavage after administration of 2.5 μg of MALP-2;

FIG. 2 shows that 2R-MALP-2 is the biologically active stereoisomer of MALP-2;

FIG. 3 shows the time dependence of leukocyte infiltration in the lungs of rats after treatment with 2R-MALP-2;

FIG. 4 shows the differential cell counts in the bronchoalveolar fluid after treatment with 2R-MALP-2; and,

FIG. 5 shows the development of lung metastases in rats topically treated with MALP-2 at different time points after tumor cell inoculation.

### DETAILED DESCRIPTION OF PREFERRED EMBODIMENTS

Lipopeptides or lipoproteins having the following general formula (I):

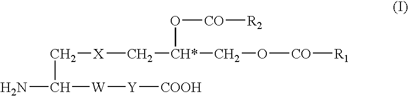

wherein:

R₁ and R₂, which may be the same or different from one another, denote C₇₋₂₅ alkyl, C₇₋₂₅ alkenyl, or C₇₋₂₅ alkynyl,

X denotes S, O, or CH₂,

W denotes CO or S(O)ₙ (where n=1 or 2),

Y denotes a physiologically acceptable amino acid sequence, and

* denotes an asymmetric carbon atom,

can be used for prevention of lung inflammations (especially in risk groups—analogously to flu protection vaccination), for increasing lymphatic tissue in the bronchial mucosa (as a result of which the effectiveness of a subsequent inhalational vaccination is improved (adjuvant effect)), for treating lung infections and lung tumors, and for treating and preventing lung metastases. While racemic mixtures of lipopeptides in accordance with formula I are effective for treating lung infections and lung tumors, the asymmetric carbon atom labeled * preferably has the absolute R configuration. When X═S (sulfur), the R configuration is particularly preferred.

For the biological activity of compounds in accordance with formula I and in the context of the methods of the invention to be conserved, the amino acid sequence is practically irrelevant provided that, the compound is adequately soluble, the biological half life is appropriate within the system, and the peptide moiety and parts thereof do not react with the specific immune system or any other physiological systems such as, for example, the complement system or the blood clotting system.

In particular, the amino acid sequence comprises about 1 amino acid residue to about 13 amino acid residues. In other embodiments, the amino acid sequence consists of one amino acid residue to 13 amino acid residues.

Furthermore, Y can denote an amino acid sequence reading, from the N-terminal to the C-terminal end, GNNDESNISFKEK, it being possible for any 1, 2, 3, 4, 5, or 6 amino acids to be deleted or exchanged, provided that the resulting lipopeptide or lipoprotein is water-soluble or amphoteric. Isofunctional amino acids, especially, may be exchanged.

For example, the amino acid sequence resulting from deletion or exchange can have a degree of homology, with respect to the starting sequence, of at least about 55%, at least about 60%, at least about 70%, at least about 80%, at least about 85%, and at least about 90%.

In formula I, the C₇₋₂₅ alkyl, C₇₋₂₅ alkenyl, or C₇₋₂₅ alkynyl can be a C₁₅ alky, C₁₅ alkenyl, or C₁₅ alkynyl, respectively. The double bond(s) of a C₇₋₂₅ alkenyl radical preferably have the cis-configuration.

In the case of an S-(2,3-bisacyloxypropyl)-cysteine peptide, the acyl group is preferably a palmitoyl group.

As specific compounds there may be mentioned, for example, S-[2,3-bispalmitoyloxy-(2RS)-propyl]cysteinyl-GNNDESNISFKEK and S-[2,3-bispalmitoyloxy-(2R)-propyl]cysteinyl-GNNDESNISFKEK.

With respect to the specific compounds set forth directly above, the “S—” indicates that the substituted propyl group is connected to the sulfur atom of the cysteine moiety.

The lipopeptides or lipoproteins of the invention may be administered in the form of an aqueous solution, a suspension, or an emulsion suitable for inhalation.

The methods of the invention can be used, for example, for the treatment of lung infections such as recurrent respiratory tract infections in chronic lung diseases, and for the treatment of lung tumors such as primary tumors of lung epithelium and lung metastases of extrapulmonary tumors. Without intending to fix on a particular theory, it is assumed that the mechanism of action is based on the recruitment and activation of immune cells, especially on the instigation of leukocyte infiltration into the lungs. In particular, the methods of the invention cause the recruitment and activation of natural killer (NK) cells and dendritic cells. NK cells are involved in the lysis of infected host cells, defense against parasitic infection, and surveillance against tumors. Dendritic cells are of primary importance for stimulating a specific immune response. See, for example, FIG. 4 which shows an increase of such cells after administration of a compound in accordance with formula I. The methods in accordance with the invention also allow for immune functions to be regained or improved after lung transplantation.

As used herein, the term “therapeutically effective amount” refers to an amount of a lipopeptide or lipoprotein which is sufficient to alleviate, ameliorate, prevent, and/or clear the symptoms and/or the pathology of a condition or disease contemplated to be treatable by the lipopeptides or lipproteins of the invention. The methods in accordance with the invention contemplate administration whether or not symptoms are manifest, i.e., prophylactic administration is contemplated. In particular, the methods can be used to prevent the dissemination of tumor cells which are generated during surgical procedures on tumors. In this regard, compounds in accordance with the invention can be administered prior to or following surgery in order to prevent metastases. Preferably, the compounds are administered immediately after surgery.

Appropriate dosages of lipopeptides or lipoproteins in accordance with the invention may be determined by monitoring leukocyte infiltration in the lungs using x-ray, computer tomography, bronchoalveolar lavage, or classical lung function tests.

### EXAMPLES

The following Examples are provided to illustrate the methods of the invention, but are not intended to limit the scope thereof. Example 1 is directed to the preparation of suitable lipopeptides for use in the methods according to the invention. Examples 2-5 are directed to treatment protocols which illustrate the efficacy of the methods of the invention.

### Example 1

Resin-bound protected peptides were synthesized as described by Morr et al. (Morr et al. 2002. Eur. J. Immunol. 32: 3337-3347). The stereoisomers of N-Fmoc-protected S-(2,3-dihydroxypropyl)-L-cysteine derivatives were synthesized as outlined by Metzger et al. (1991) (see Metzger, J. W., K-H. Wiesmüller and G. Jung. 1991. Int. J. Peptide Protein. Res. 38: 545-554), and coupled to the resin-bound protected peptides as described in Metzger et al. (1995) (see Metzger et al. 1995. J. Peptide Science 3:184-190). Protecting groups were cleaved and the lipopeptide was removed from the resin as described by Morr et al.

The biologically active enantiomer of MALP-2, i.e., S-[2,3-bispalmitoyloxy-(2R)-propyl]cysteinyl-GNNDESNISFKEK, was synthesized as described by Morr et al., using (R)-(+)-oxirane-2-methanol (Fluka, Switzerland) as starting material for the lipid moiety. Crude MALP-2 was further purified in 10-mg batches by reversed phase HPLC on a SP 250/10 Nucleosil 300-7 C8 column (Macherey & Nagel, Düren, Germany) and was eluted at 40° C. with a linear water/2-propanol gradient containing 0.1% trifluoroacetic acid. Elution of active material was monitored by a nitric oxide release assay (see Mühlradt, P. F., M. Kieβ, H. Meyer, R. Süβmuth, and G. Jung. 1997. J. Exp. Med. 185:1951-1958). The exact peptide content of the final product was determined by amino acid analysis. The final product was also characterized by mass spectroscopy.

At page 547 of the stereospecific synthesis described by Metzger et al. (1991), “a” is defined to be the diastereomer of a glyceryl moiety having the R configuration at the C-2 position, and “b” is defined to be the diastereomer of the same having the S configuration. The authors incorrectly indicate that compounds having the R configuration are synthesized using (S)-(−)-glycidol (an alternative name is (S)-(−)-oxirane-2-methanol) as starting material. Compounds having the R configuration are synthesized using (R)-(+)-oxirane-2-methanol (Fluka, Switzerland) as starting material, as indicated in this Example.

### Example 2

In Examples 2-4, male Lewis rats and brown Norway rats with a mean body weight of 259±56 g were obtained from the central animal laboratory (Medical School of Hannover, Hannover, Germany). They were maintained under specific-pathogen-free conditions until administration of compounds in accordance with the invention.

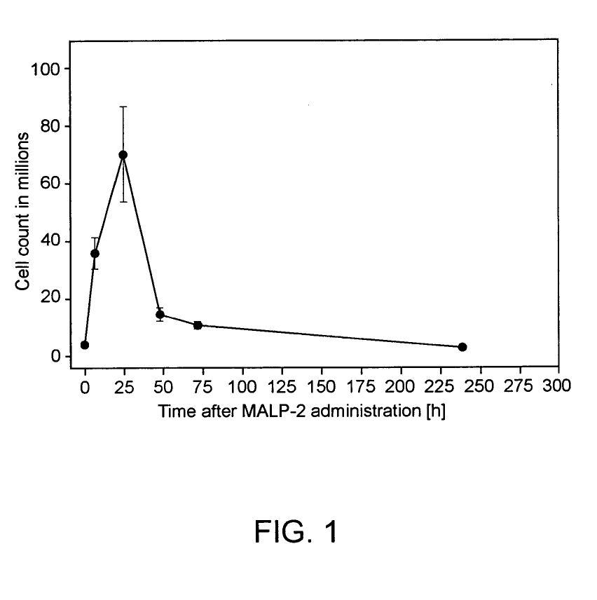

3 μg of MALP-2 (an S-(2,3-bisacyloxypropyl)-cysteine peptide) were instilled into the trachea of rats. At various times after treatment, cells from the lung lumen were determined by means of bronchoalveolar lavage (BAL). After a few hours, the MALP-2 treatment resulted in an increase in the number of leukocytes in the respiratory tracts and the alveolar space of the animals, which lasts for a few days (see FIG. 1). An adverse effect on the animals as a result of the MALP-2 administration was not detectable. Food consumption, bodyweight, activity and behavior were not noteworthy.

### Example 3

With the aid of a mask, rats were made to inhale 30 μg doses of MALP-2 on six occasions at one-week intervals. This resulted in an increase in the lymphatic tissue in the bronchial mucosa. In this test, too, the animals' food consumption, bodyweight, activity and behavior were clinically not noteworthy.

Histological investigation showed that three days after intratracheal instillation of 25 μg of 2R-MALP-2 there was a multifocal, pneumonia-like infiltration consisting of neutrophils and mononuclear cells. Inflamatory cells were found around most of the larger and the smaller vessels and bronchi. In addition, neutrophils were present in many alveoli, indicating alveolitis. After application of 2.5 μg of 2R-MALP-2, a mild inflammation was observed. The lung morphology of animals treated with the control vehicle or the S-isomer were not altered.

### Example 4

2-R-MALP-2

In the majority of experiments, the biologically active, chemically synthesized 2-R-MALP-2 stereoisomer, corresponding to the natural compound, was used for intracheal administration. In some experiments, the inactive 2-S-MALP-2 stereoisomer was used to check for nonspecific effects of lipopeptides. Both compounds were synthesized and purified as described in Example 1, and kept as 1 mg/ml stock solutions in a 33 vol. % 2-propanol, 67 vol. % water mixture. Stock solutions were diluted with pyrogen-free 0.9 wt. % sodium chloride solutions (Braun, Melsungen, Germany) shortly before application. Solvent controls consisted of corresponding mixtures without MALP-2.

Intrapulmonary Application

Intratracheal administration of the compounds was performed under short ether anesthesia. During this process, rats were suspended in a hanging position by a rubber band fixed to the rats' upper jaw teeth. After intubation of the trachea via the oral cavity, a total volume of 500 μl was administered. Before administration, the correct position of the tube was checked by blowing air into the lung.

Preparation of Bronchoalveolar Cells

The animals' lungs were lavaged 10 times with 5 ml of cold 0.9 wt. % NaCl solutions via a tracheal cannula. The lavage fluid was pooled, and an average of >90% of the volume was recovered. The bronchoalveolar (BAL) cells were processed for blood leukocytes as described herein.

Staining of Lymphocyte Subsets, Neutrophils, Monocytes/Macrophanges, and Dendritic Cells

The total cell numbers in the lung compartments (BAL, marginal pool, and interstitium) and in the blood were determined in a Neubauer counting chamber. Differential cell counts were assessed on cytospins. The slides were fixed in acetone for 10 min and washed with Tris-buffered saline containing 0.1 wt. % Tween 20. Cells were stained with a monoclonal antibody cocktail against lymphocytes (R73 for T cells, O×12 for B cells, and 3.2.3 for natural killer cells [all cells available from Serotec, Oxford, United Kingdom]). At least 400 cells were differentiated on each slide. In addition, neutrophils and macrophages were morphologically identified by May-Grünwald and/or Giemsa staining.

For flow cytometry, cells were transferred into microtiter plates (10⁶ cells/well) and washed twice in PBS containing 1% BSA and 0.1% NaN₃. The staining was performed with the appropriate primary antibodies described below. After the cells were washed twice, the secondary antibody was incubated for 30 min at 4° C. The cells were analyzed by using a FACScan flow cytometer (Becton Dickinson, Mountain View, Calif.), focusing on the lymphocyte cluster. CD8⁺ T cells were identified as R73⁺ O×8⁺; CD4⁺ T cells were identified as R73⁺ O×8⁻; memory CD4⁺ T cells were identified as O×22⁻ W3/25^{high}; naive T cells were identified as O×22⁺ W3/25^{high}; and monocytes were identified as O×22⁻ W3/25^{low}. Dendritic cells were identified as low-autofluorescence cells, non-T and non-B lymphocytes, and double positive for O×6 and O×62 (Lambrecht et al., 1999. Am. J. Respir. Cell. Mol. Biol. 20:1165-1174). All primary antibodies were monoclonal mouse anti-rat (Serotec). Unconjugated antibodies were detected by using phycoerythrin-conjugated secondary antibody (_{K}PE; Dianova, Hamburg, Germany) and biotinylated primary antibodies with Red 670-streptavidin (Gibco, Gaithersburg, Md.). Isotype-matched antibodies served as a control.

Leukocyte Infiltration in Response to Two Stereoisomers of MALP-2

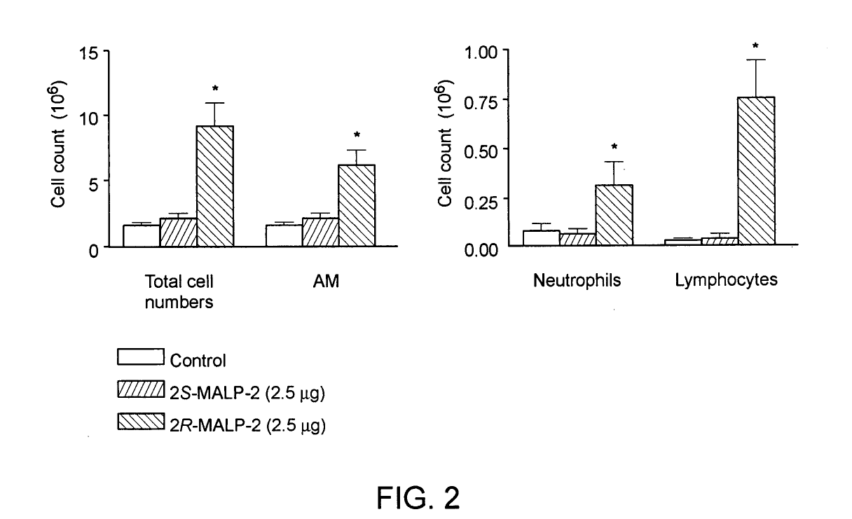

To investigate whether the lipopeptide MALP-2 would be able to exert effects in the lungs, 2.5 μg of the natural stereoisomer 2R-MALP-2 was instilled into the lungs of anesthetized rats. Rats treated with 2.5 μg 2S-MALP-2, which has shown poor in vitro activity (Morr et al. 2002. Eur. J. Immunol. 32: 3337-3347), or with 2.5 μg of the control vehicle (33 vol. % 2-propanol, 67 vol. % water mixture appropriately diluted with saline (0.9 wt. % NaCl solution) served as controls. FIG. 2 shows that three days after intratracheal application, the total cell number in the BAL was clearly increased only in the group of animals that had received 2R-MALP-2 when compared with those that had received 2S-MALP-2 or vehicle control. FIG. 2 also illustrates that a significant increase of alveolar macrophages (AM), neutrophils, and lymphocytes was observed in the 2R-MALP-2 treated animals. Taken together, only the natural 2R stereoisomer of MALP-2 specifically affected the cell counts and distribution of subsets in the BAL. There was no nonspecific response to lipopeptide or to the traces of organic solvent in the vehicle control.

With respect to FIG. 2, the data are means±the standard error of the mean (SEM; n=six per group). The asterisks indicate significant differences (P<0.01) from the vehicle control (which did not contain MALP-2) and the inactive stereoisomer 2S-MALP-2 as calculated by the Mann-Whitney U test.

Time Dependency of Leukocyte Accumulation in Response to 2R-MALP-2

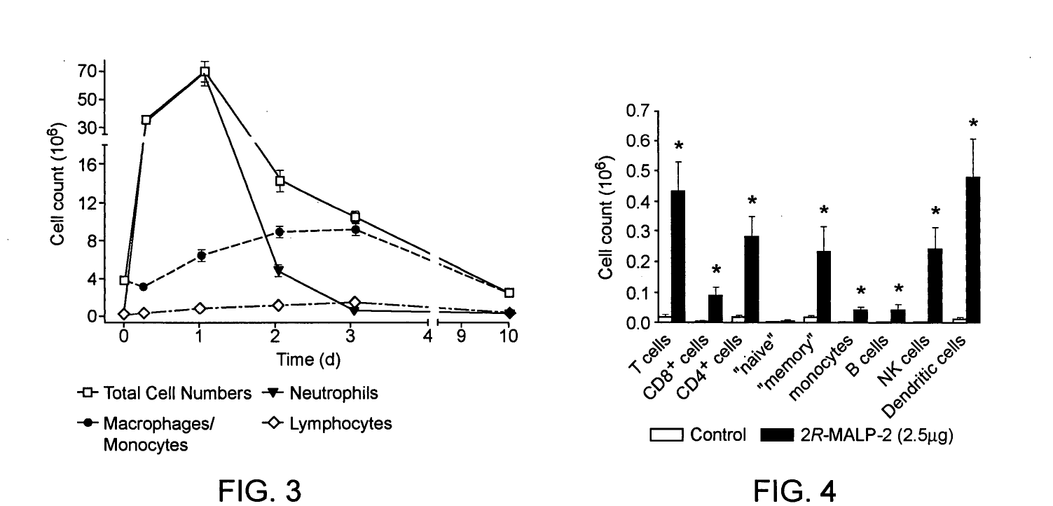

FIG. 3 shows that the maximal cell count in the BAL after the application of 2.5 μg of 2R-MALP-2 was found after 24 h, and that six hours after treatment the total cell number had already increased about 10-fold. The cellular accumulation in the bronchoalveolar space at this early time point was mainly caused by neutrophils. After 24 h the cells still mainly consisted of neutrophils but now with a significant percentage of AM. In the following days neutrophil counts decreased, although the AM count continued to increase. Lymphocyte numbers, although they amounted to only a few percent of the total BAL cells, were significantly increased at 24 and 48 h (0 h, [0.04±0.01]×10⁶; 24 h, [0.6±0.1]×10⁶; 48 h, [0.9±0.1]×10⁶). Maximal lymphocyte accumulation was achieved 72 h after instillation ([1.2±0.1×10⁶). After 10 days, all investigated cell types returned to control levels.

With respect to FIG. 3, rats were sacrificed at the indicated time points after instillation of 2.5 μg of 2R-MAPL-2, and leukocytes in the bronchoalveolar lavage (BAL) were determined as described. The data are means±the SEM (n=5 per group).

Differential Cell Counts in the BAL Fluid and Other Compartments of the Lung after 2R-MALP-2 Treatment

Three days after 2R-MALP-2 application, when neutrophil counts had subsided, rats were sacrificed and minor leukocyte subsets in the BAL were determined by flow cytometry with specific antibody staining. FIG. 4 shows an increase of all subpopulations was significant after 2R-MALP-2 administration, the only exception being the “naive” CD4⁺ lymphocyte subset. The highest relative increase in cell numbers was found in T lymphocytes, NK cells, and dendritic cells, whereas the increase in B cells and monocytes was lower, but still significant.

With respect to FIG. 4, data are means±the SEM (n=8 per group). The asterisks indicated significant differences (P<0.01) from the control vehicle as calculated by the Mann-Whitney U test.

### Example 5

Animals, Injection of Tumor Cells, and Processing of Lungs

F344 rats were obtained from a breeding colony kept in barrier-reared conditions at the Central Animal Laboratory at the Medical School of Hannover, Germany. Rats were kept in a specific pathogen-free facility at 25° C. under a 12-h light/12-h dark cycle (lights on at 7:00 A.M.), with ad libitum access to food and water. For the experiments age-matched 8-wk-old male rats were used. All research and animal care procedures were approved by the Lower Saxony district government (Hannover, Germany) and followed principles described in the European Community's Council Directive of Nov. 24, 1986 (86/609/EEC).

Cell culture, injection of tumor cells, dissection of the animals, and immunohistochemistry were conducted as previously described (Tschernig et al., 1997. Am. J. Respir. Cell. Mol. Biol. 17:414-421). In brief, 1×10⁶ mammary adenocarcinoma tumor cells (MADB106 tumor cells) derived from the log phase of tumor growth were injected via the lateral tail vein, and lungs were removed at different time points thereafter. The 9-10 dimethyl-1-2-benzanthracene-induced MADB106 mammary adenocarcinoma syngeneic tumor is a selected variant cell line obtained from a pulmonary metastasis produced by the intravenous injection of the MADB100 parental adenocarcinoma induced in F344 rats (Barlozzari et al., 1985. J. Immunol. 134:2783-2789). As the tumor is originally derived from the metastatic tissue itself, the potential to seed in the lungs can be anticipated and is furthermore supported by previous experiments (von Hörsten et al., 2000. J. Immunol. Meth. 239:25-34). For in situ quantification of tumor cells at 60 min after injection, cells were vital dye stained using the fluorescein derivate 5-(and 6-) carboxyfluorescein diacetate succinimdyl ester (CFSE; Molecular Probes, Eugene, Oreg.) before injection (von Hörsten et al., 2000. J. Immunol. Meth. 239:25-34). For quantification of lung surface colonies at later time points (3 wk after tumor cell inoculation), en bloc dissected lungs and the heart were injected with 8 ml Bouin's solution (72 vol. % saturated picric acid solution, 23 vol. % formaldehyde solution (35 wt. % formaldehyde in water), and 5 vol. % glacial acetic acid) and fixed in the same solution until subpleural lung nodules were counted.

Intratracheal Administration of MALP-2

MALP-2 was synthesized as in Example 1 and kept as a stock solution of 1 mg/ml in water: 2-propanol (1:1) (vol/vol) at 4° C. For in vivo use, MALP-2 was diluted with isotonic saline for injection (Fresenius, Bad Homburg, Germany). The rats were anesthetized by isoflurane and placed into a vertical position with the heads upwards. A blunt cannula was inserted into the trachea via the mouth, and 1% MALP-2 solution (250 μl/rat) or an equal volume of saline (sham control) was instilled intratracheally, followed by 1 ml air. The animals were held in this position until they recovered from the anesthesia. MALP-2 was instilled at three different time points; i.e., simultaneously with tumor inoculation and one day or three days afterwards.

Quantification of Lung Metastasis

Counting of lung metastases was conducted according to the method described by Wexler (Wexler, 1966. J. Natl. Cancer Inst. 36:641-645). Subpleural lung surface colonies were identified due to a light, white appearance. Three weeks after inoculation, surface metastases are 1-8 mm³, mushroom-shaped, distinctly separated, and raised above the lung surface. Visible surface metastases were quantified on randomly selected lung surface areas using a gauge (1.0 cm²). Three areas per animal were examined, and subpleural lung surface colony numbers were expressed as mean/cm².

Effects of Intratracheal MALP-2 Administration on Lung Metastasis in F344 Rats

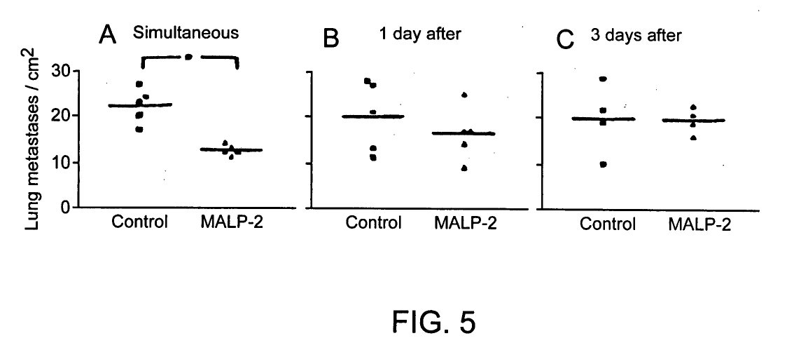

As shown in FIG. 5, lung metastasis depends on the time point of MALP-2 instillation after tumor cell inoculation. Each symbol in FIG. 5 represents one rat. A significant effect of treatment was seen when MALP-2 was administered simultaneously with tumor cell inoculation, which resulted in a 44% reduction of lung metastasis compared with sham-injected animals (F[1.9]=30.0; P=0.0006; FIG. 5A). In contrast, no effects on lung colonization of tumor cells were found when MALP-2 was given one or three days after tumor cell injection (as shown in FIGS. 5B and 5C).

With regards to FIG. 5, saline was used in the control animals, and the horizontal line represents mean value. The variability in the controls is most likely due to a differential endogenous stress response in these rats. However, when MALP-2 is administered, additional immunodulatory effects might interfere with and even overrule these stress-dependent effects.

What is claimed is:

1. A method of treating and preventing lung metastases of extrapulmonary tumors comprising:
administering a therapeutically effective amount of a lipopeptide or lipoprotein to an individual, wherein the lipopeptide or lipoprotein has the following structure: 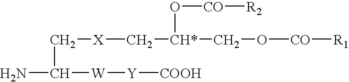
wherein:
R₁ and R₂, which may be the same or different from one another, denote C₇₋₂₅ alkyl, C₇₋₂₅ alkenyl, or C₇₋₂₅ alkynyl,
X denotes S, O, or CH₂,
W denotes CO or S(O)ₙ (where n=1 or 2),
Y denotes a physiologically acceptable amino acid sequence, and
* denotes an asymmetric carbon atom.

2. The method according to claim 1, wherein the asymmetric carbon atom labeled * has the absolute R configuration.

3. The method according to claim 1, wherein Y comprises about one amino acid residue to about 13 amino acid residues.

4. The method according to claim 1, wherein Y denotes an amino acid sequence reading, from the N-terminal to the C-terminal end, GNNDESNISFKEK, it being possible for any 1, 2, 3, 4, 5, or 6 amino acids to be deleted or exchanged provided that the resulting lipopeptide or lipoprotein is water-soluble or amphoteric.

5. The method according to claim 4, wherein the resulting amino acid sequence has a degree of homology, with respect to the starting sequence, of at least about 55%.

6. The method according to claim 1, wherein the C₇₋₂₅ alkyl is a C₁₅ alkyl, the C₇₋₂₅ alkenyl is a C₁₅ alkenyl, or the C₇₋₂₅ alkynyl is a C₁₅ alkynyl.

7. The method according to claim 1, wherein the double bond(s) of the C₇₋₂₅ alkenyl have the cis-configuration.

8. The method according to claim 1, wherein the lipopeptide or lipoprotein is S-[2,3-Bispalmitoyloxy-(2RS)-propyl]cysteinyl-GNNDESNISFKEK.

9. The method according to claim 1, wherein the lipopeptide or lipoprotein is S-[2,3-Bispalmitoyloxy-(2R)-propyl]cysteinyl-GNNDESNISFKEK.

10. The method according to claim 1, wherein the lipopeptide or lipoprotein is in the form of an aqueous solution, suspension, or emulsion that is suitable for inhalation.

11. The method according to claim 1, wherein the individual is a mammal.

12. A method of treating lung infections and lung tumors, except lung metastases of extrapulmonary tumors, comprising:
administering a therapeutically effective amount of a lipopeptide or lipoprotein to an individual, wherein the lipopeptide or lipoprotein has the following structure: 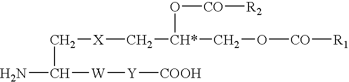
wherein:
R₁ and R₂, which may be the same or different from one another, denote C₇₋₂₅ alkyl, C₇₋₂₅ alkenyl, or C₇₋₂₅ alkynyl,
X denotes S, O, or CH₂,
W denotes CO or S(O)n (where n=1 or 2),
Y denotes a physiologically acceptable amino acid sequence, and
* denotes an asymmetric carbon atom.

13. The method according to claim 12, wherein the asymmetric carbon atom labeled * has the absolute R configuration.

14. The method according to claim 12, wherein Y comprises about one amino acid residue to about 13 amino acid residues.

15. The method according to claim 12, wherein Y denotes an amino acid sequence reading, from the N-terminal to the C-terminal end, GNNDESNISFKEK, it being possible for any 1, 2, 3, 4, 5, or 6 amino acids to be deleted or exchanged provided that the resulting lipopeptide or lipoprotein is water-soluble or amphoteric.

16. The method according to claim 15, wherein the resulting amino acid sequence has a degree of homology, with respect to the starting sequence, of at least about 55%.

17. The method according to claim 12, wherein the C₇₋₂₅ alkyl is a C₁₅ alkyl, the C₇₋₂₅ alkenyl is a C₁₅ alkenyl, or the C₇₋₂₅ alkynyl is a C₁₅ alkynyl.

18. The method according to claim 12, wherein the double bond(s) of the C₇₋₂₅ alkenyl have the cis-configuration.

19. The method according to claim 12, wherein the lipopeptide or lipoprotein is S-[2,3-Bispalmitoyloxy-(2RS)-propyl]cysteinyl-GNNDESNISFKEK.

20. The method according to claim 12, wherein the lipopeptide or lipoprotein is S-[2,3-Bispalmitoyloxy-(2R)-propyl]cysteinyl-GNNDESNISFKEK.

21. The method according to claim 12, wherein the lipopeptide or lipoprotein is in the form of an aqueous solution, suspension, or emulsion that is suitable for inhalation.

22. The method according to claim 12, wherein the individual is a mammal.

23. The method according to claim 12, wherein the lung infections are recurrent respiratory tract infections in chronic lung diseases and
the lung tumors are primary tumors of lung epithelium.
